(19) United States
(12) Patent Application Publication
(10) Pub. No.: US 2011/0295037 A1
(43) Pub. Date: Dec. 1, 2011

(54) PROCESSES FOR PREPARING CINACALCET HYDROCHLORIDE AND POLYMORPHIC FORMS THEREOF

(75) Inventors: Tibor Szekeres, Budapest (HU); Jozsef Repasi, Budapest (HU); Andras Szabo, Budapest (HU); Monica Benito Velez, Buenos Aires (ES); Bernardino Mangion, Santa Lucia (MT)

(73) Assignee: MEDICHEM, S.A., BARCELONA (ES)

(21) Appl. No.: 12/303,912

(22) PCT Filed: Jun. 8, 2007
(86) PCT No.: PCT/IB07/04309

§ 371 Date: Oct. 4, 2010

Related U.S. Application Data
(60) Provisional application No. 60/811,782, filed on Jun. 8, 2006.

(51) Int. Cl. C07C 209/60; C07C 211/30

(52) U.S. Cl. 564/387

(57) ABSTRACT

The invention relates to cinacalcet hydrochloride, new polymorphic crystalline forms of cinacalcet hydrochloride, amorphous cinacalcet hydrochloride and synthetic processes for their preparation.

### CROSS REFERENCE TO RELATED APPLICATIONS

This application claims priority to U.S. Provisional Application No. 60/811,782, filed Jun. 8, 2006, application which is expressly incorporated herein by reference in its entirety.

### BACKGROUND OF THE INVENTION

1. Field of the Invention

The invention relates to cinacalcet hydrochloride, new polymorphic crystalline forms of cinacalcet hydrochloride, amorphous cinacalcet hydrochloride and synthetic processes for their preparation.

2. Discussion of the Related Art

Cinacalcet hydrochloride is a commercially marketed pharmaceutically active substance known to be useful for the treatment of hyperparathyroidism and the preservation of bone density in patients with kidney failure or hypercalcemia due to cancer. Cinacalcet hydrochloride is the generic international denomination for N-[1-(R)-(−)-(1-naphthyl)ethyl]-3-[3-(trifluoro methyl)phenyl]-1-aminopropane hydrochloride, which has the formula (I) given below:

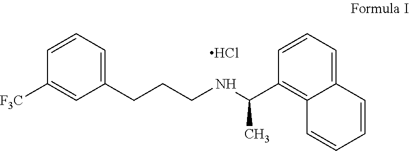

Cinacalcet hydrochloride is an oral calcimimetic drug. In the United States, it is marketed under the name Sensipar® and, in Europe, it is marketed under the name Mimpara® and Parareg®. It has been approved for the treatment of secondary hyperparathyroidism in patients with chronic kidney disease on dialysis and for the treatment of hypercalcemia in patients with parathyroid carcinoma.

U.S. Pat. No. 6,011,068 generally describes cinacalcet and its pharmaceutically acceptable acid additions salts but does not provide any examples for the preparation of the same.

U.S. Pat. No. 6,211,244 describes cinacalcet and its pharmaceutically acceptable acid chloride addition salt but does not provide any examples for the preparation of cinacalcet and/or cinacalcet hydrochloride.

Drugs 2002, 27(9), 831-836 discloses a synthetic scheme for preparing cinacalcet hydrochloride according to the general procedure described in U.S. Pat. No. 6,211,244. This disclosed synthetic route is illustrated in Scheme 1, below. This synthetic route, however, uses a titanium isopropoxide catalyst. In this regard, metal catalysts are disfavored for industrial implementation.

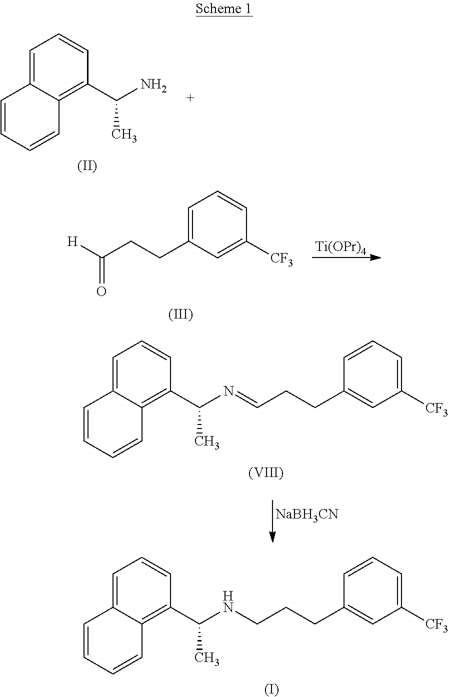

Apart from the synthetic route illustrated in Scheme 1 above, no specific example for the preparation of cinacalcet hydrochloride has been reported in the literature. Hence, there is a need in the art for a process for preparing cinacalcet and its salts for industrial scale, and which avoids the use of Ti(OiPr)₄ as catalyst.

International Patent Publication No. WO 2006/127933 discloses that the crystalline cinacalcet hydrochloride currently marketed as Sensipar® is characterized as crystalline Form I (denominated as Form I), and encompasses processes for its preparation. Further, International Patent Publication No. WO 2006/127941 relates to amorphous cinacalcet hydrochloride and to a process for its preparation.

Polymorphism is very common among pharmaceutical substances. It is commonly defined as the ability of any substance to exist in two or more crystalline phases that have a different arrangement and/or conformation of the molecules in the crystal lattice. Different polymorphs differ in their physical properties such as melting point, solubility, chemical reactivity, etc. These can appreciably influence pharmaceutical properties such as dissolution rate and bioavailability.

The discovery of new crystalline forms provides opportunities to improve the characteristics of a pharmaceutical product. Hence, there is a need for stable, well-defined and reproducible new crystalline forms of cinacalcet hydrochloride.

### SUMMARY OF THE INVENTION

The invention provides a process for preparing cinacalcet, its salts and/or solvates thereof. In particular, the invention provides a process for preparing cinacalcet, its salts and/or solvates thereof which includes the reductive amination, in the absence of titanium isopropoxide, of 3-(3-trifluoromethylphenyl)propanal (Compound III) with (R)-(1-naphthyl)ethylamine (Compound II) to yield cinacalcet, and optionally converting the cinacalcet into one of its corresponding salts and/or solvates thereof. Preferably, the produced cinacalcet is converted to its hydrochloride salt.

Another aspect of the invention includes cinacalcet, its salts and/or solvates having a high degree of chemical and optical purity.

Surprisingly it has now been found that cinacalcet hydrochloride can exist in at least two novel crystalline forms.

The invention includes new crystalline forms of cinacalcet hydrochloride, designated herein as cinacalcet hydrochloride Forms II and III methods of making the same and formulations of the same.

The invention further includes methods of making cinacalcet hydrochloride Form I and amorphous form.

Another aspect of the invention is cinacalcet hydrochloride Form I with a high degree of chemical and optical purity.

In another aspect, the invention provides a process for preparing cinacalcet hydrochloride Form I, generally comprising:

a. dissolving cinacalcet hydrochloride in an organic solvent;

b. removing the solvent;

c. recovering cinacalcet hydrochloride; and

d. drying the cinacalcet hydrochloride,

wherein the solvent is at least one of an alcoholic solvent, a ketonic solvent, dichloromethane, an ester solvent, an ether solvent, an aprotic solvent or mixtures thereof.

In another aspect, the invention provides a process for preparing cinacalcet hydrochloride Form I, generally comprising:

a. obtaining cinacalcet hydrochloride by recrystallization from a solvent; and

b. drying the cinacalcet hydrochloride,

wherein the solvent is at least one of an alcoholic solvent, a ketonic solvent, an ester solvent, an ether solvent, a hydrocarbon solvent, an aprotic solvent, water or mixtures thereof.

In another aspect, the invention provides a process for preparing cinacalcet hydrochloride Form I, generally comprising

a. treating cinacalcet hydrochloride in an organic solvent;

b. recovering the crystalline form as a precipitate; and

c. drying the crystalline form of cinacalcet hydrochloride,

wherein the solvent is at least one of water, ethanol or mixtures thereof.

In another aspect, the invention provides a process for preparing cinacalcet hydrochloride Form I, generally comprising:

a. dissolving cinacalcet hydrochloride in an first organic solvent

b. optionally filtering the obtained solution,

c. adding a second solvent, and

d. recovering the crystalline form as a precipitate,

wherein the first organic solvent is at least one of an alcoholic solvent, a ketonic solvent, a chlorinated solvent, an ether solvent or mixtures thereof and the second solvent is at least one of an ether solvent, a hydrocarbon solvent, water or mixtures thereof.

In another aspect, the invention provides a novel crystalline form of cinacalcet hydrochloride, herein described as Form II.

Another aspect of the invention is cinacalcet hydrochloride Form II with a high degree of chemical and optical purity.

In another aspect, the invention provides a process for preparing cinacalcet hydrochloride Form II, generally comprising:

a. dissolving cinacalcet hydrochloride in chloroform;

b. removing the chloroform;

c. recovering cinacalcet hydrochloride; and

d. drying the cinacalcet hydrochloride.

In another aspect, the invention provides a process for preparing cinacalcet hydrochloride Form II, generally comprising

a. suspending cinacalcet hydrochloride in an organic solvent;

b. filtering the obtained solid;

c. recovering cinacalcet hydrochloride; and

d. drying the cinacalcet hydrochloride,

wherein the organic solvent is a chlorinated solvent.

In another aspect, the invention provides a novel crystalline form of cinacalcet hydrochloride, herein described as Form III.

Another aspect of the invention is cinacalcet hydrochloride Form III with a high degree of chemical and optical purity.

In another aspect, the invention provides processes for preparing cinacalcet hydrochloride Form III, generally comprising:

a. dissolving cinacalcet hydrochloride in chloroform,

b. adding a second solvent;

c. recovering the crystalline form as a precipitate; and

d. drying the crystalline form of cinacalcet hydrochloride,

wherein the second solvent is at least one of an ether solvent, a hydrocarbon solvent or mixtures thereof.

Another aspect of the invention is amorphous cinacalcet hydrochloride with a high degree of chemical and optical purity.

In another aspect, the invention provides processes for preparing amorphous cinacalcet hydrochloride, generally comprising:

a. dissolving cinacalcet hydrochloride in an organic solvent;

b. removing the solvent;

c. recovering the amorphous form as a precipitate; and

d. drying the amorphous form of cinacalcet hydrochloride,

wherein the organic solvent is at least one of an alcoholic solvent, a chlorinated solvent, an ether solvent, a hydrocarbon solvent or mixtures thereof.

The invention further includes cinacalcet hydrochloride having a particle size distribution wherein approximately 85-95% of the total volume is made of particles having a diameter of approximately 283 μm or below, preferably approximately 85-95% of the total volume is made of particles having a diameter of approximately 80 μm or below, more preferably approximately 85-95% of the total volume is made of particles having a diameter of approximately 35 μm or below.

The invention further includes cinacalcet hydrochloride having a surface area of approximately 0.6 to approximately 2.7 m²/g.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1 illustrates the X-ray powder diffractogram (XRD) of cinacalcet hydrochloride Form I obtained in Example 1;

FIG. 2 illustrates the Infrared (IR) spectrum of cinacalcet hydrochloride Form I obtained in Example 1;

FIG. 3 illustrates the X-ray powder diffractogram (XRD) of cinacalcet hydrochloride Form II obtained in Example 7;

FIG. 4 illustrates the X-ray powder diffractogram (XRD) of cinacalcet hydrochloride Form III obtained in Example 12;

FIG. 5 illustrates the Thermagravimetric analysis thermogram (TGA) of cinacalcet hydrochloride Form III obtained in Example 13;

FIG. 6 illustrates the X-ray powder diffractogram (XRD) of amorphous cinacalcet hydrochloride obtained in Example 13; and

FIG. 7 illustrates the Infrared (IR) spectrum of Cinacalcet hydrochloride amorphous obtained in Example 13.

### DETAILED DESCRIPTION OF THE PREFERRED EMBODIMENTS

Reference will now be made in detail to the preferred embodiments of the invention. This invention may, however, be embodied in many different forms and should not be construed as limited to the embodiments set forth herein.

The invention provides a process for preparing cinacalcet, its salts and/or solvates thereof.

More particularly, the invention provides a process for preparing cinacalcet, its salts and/or solvates thereof which includes the reductive amination, in the absence of titanium isopropoxide, of 3-(3-trifluoromethyl phenyl)propanal (Compound III) with (R)-(1-naphthyl)ethylamine (Compound II) to yield cinacalcet and optionally converting the cinacalcet into one of its corresponding salts and/or solvates thereof. Preferably, the cinacalcet produced is converted to its hydrochloride salt.

Preferably Compound II is of high optical purity (e.g., greater than 99.5% enantiomeric excess) when used in the above-described process.

Preferably the reducing agent is sodium triacetoxyborohydride.

The resulting cinacalcet salts and/or solvates obtained by the method described above have a high degree of chemical and optical purity, according to high performance liquid chromatography (HPLC). In one embodiment of the invention, cinacalcet salts and/or solvates of the invention have a degree of chemical purity in the range of about 99.00% to about 99.95% and an optical purity in the range of about 99.0 to about 100%. In another embodiment of the invention, cinacalcet salts and/or solvates of the invention have a degree of chemical purity in the range of about 99.60% to about 99.80% and an optical purity of about 99.90% to about 100%.

The invention includes new crystalline forms of cinacalcet hydrochloride (designated herein as cinacalcet hydrochloride Forms II and III), methods of making the same and formulations of the same.

The invention further includes methods of making cinacalcet hydrochloride Form I and amorphous form.

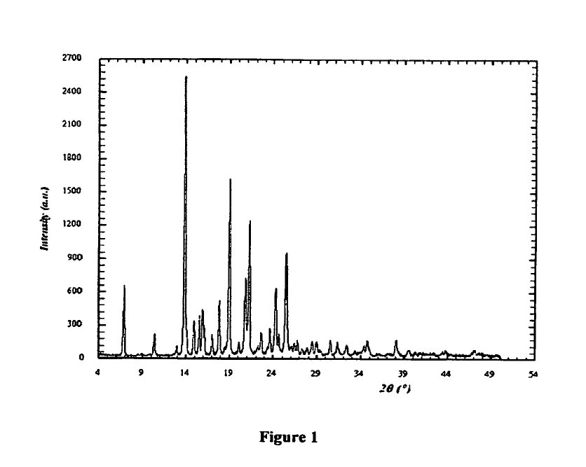

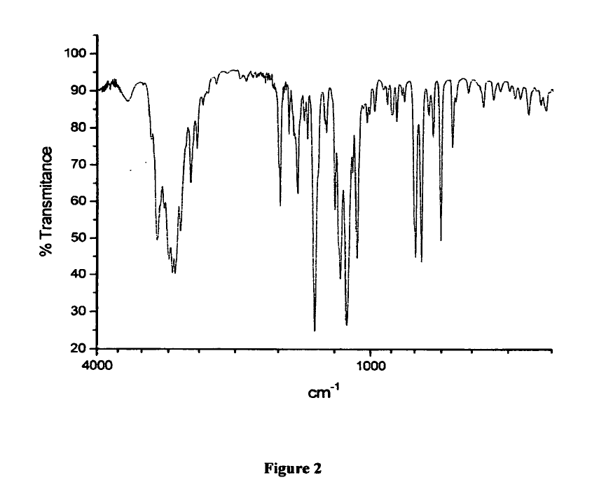

Cinacalcet hydrochloride Form I is characterized by its XRD pattern (2θ) (±0.2°) having characteristics peaks at approximately 6.9°, 10.4°, 13.8°, 15.5°, 17.8°, 19.0°, 21.2°, 24.2° and 25.4°. FIG. 1 illustrates the XRD of cinacalcet hydrochloride Form I. FIG. 2 illustrates the infrared spectrum of cinacalcet hydrochloride Form I which has its main peaks at 3051, 2966, 2864, 2796, 2750, 2712, 2642, 2513, 2430, 1587, 1518, 1450, 1402, 1379, 1327, 1252, 1167, 1128, 1072, 1018, 980, 922, 899, 878, 845, 799, 775, 731, 704 and 664 cm⁻¹. Cinacalcet hydrochloride Form I is further characterized by having a high chemical and optical purity, according to high performance liquid chromatography (HPLC), a low residual solvent content and is generally free of insoluble materials/compounds.

In one embodiment of the invention, cinacalcet hydrochloride Form I has a degree of chemical purity in the range of about 99.00% to about 99.95% and an optical purity in the range of about 99.0 to about 100%. In another embodiment of the invention, cinacalcet hydrochloride Form I has a degree of chemical purity in the range of about 99.60% to about 99.80% and an optical purity of about 99.90% to about 100%.

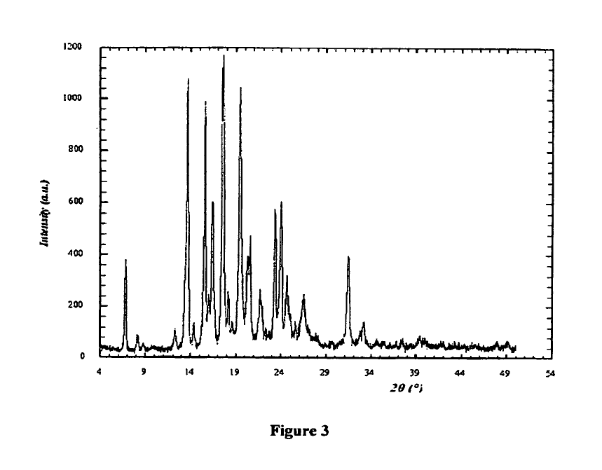

Cinacalcet hydrochloride Form II is characterized by its XRD pattern (2θ) (±0.2°) having characteristics peaks at approximately 13.7°, 14.3°, 16.6°, 17.5°, 19.4°, 20.3°, 20°.6, 23.3° and 31.4°. FIG. 3 illustrates the XRD of cinacalcet hydrochloride Form II. Cinacalcet hydrochloride Form II is further characterized by having a high chemical and optical purity, according to high performance liquid chromatography (HPLC), a low residual solvent content and is generally free of insoluble materials/compounds.

In one embodiment of the invention, cinacalcet hydrochloride Form II has a degree of chemical purity in the range of about 99.00% to about 99.95% and an optical purity in the range of about 99.0 to about 100%. In another embodiment of the invention, cinacalcet hydrochloride Form II has a degree of chemical purity in the range of about 99.60% to about 99.80% and an optical purity of about 99.90% to about 100%.

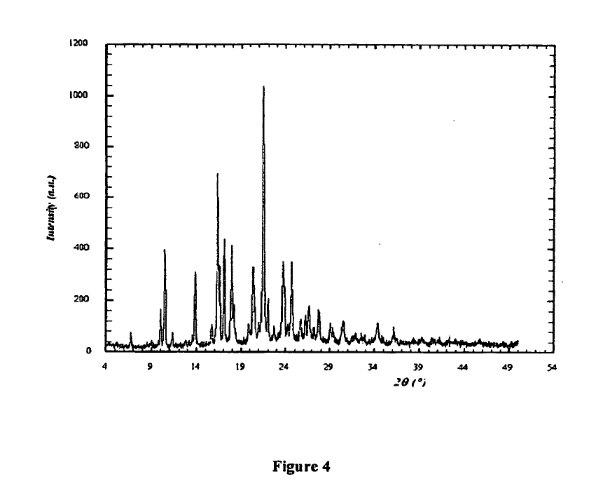

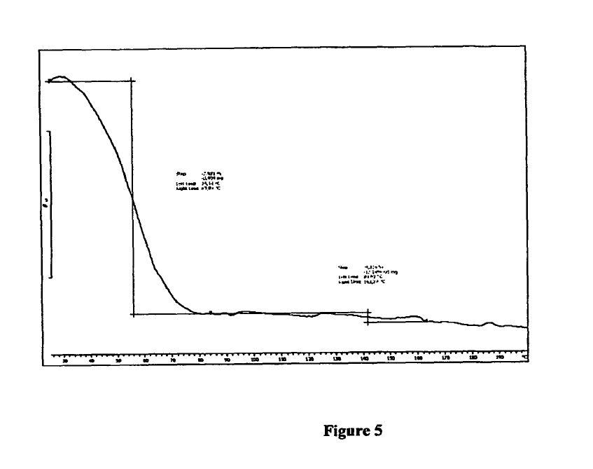

Cinacalcet hydrochloride Form III is characterized by its XRD pattern (2θ) (±0.2°) having characteristics peaks at approximately 10.0°, 10.5°, 16.2°, 17.0°, 17.8°, 20.2°, 21.5° and 23.6°. FIG. 4 illustrates the XRD of cinacalcet hydrochloride Form III. Cinacalcet hydrochloride Form III is further characterized by being a chloroform solvate. FIG. 5 illustrates the thermogravimetric analysis thermogram (TGA) of cinacalcet hydrochloride Form III. Cinacalcet hydrochloride Form III is further characterized by having a high chemical and optical purity, according to high performance liquid chromatography (HPLC) and is generally free of insoluble materials/compounds.

In one embodiment of the invention, cinacalcet hydrochloride Form III has a degree of chemical purity in the range of about 99.00% to about 99.95% and an optical purity in the range of about 99.0 to about 100%. In another embodiment of the invention, cinacalcet hydrochloride Form III has a degree of chemical purity in the range of about 99.60% to about 99.80% and an optical purity of about 99.90% to about 100%.

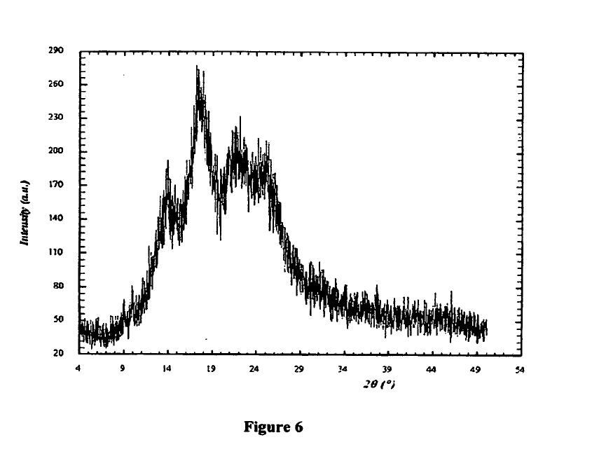

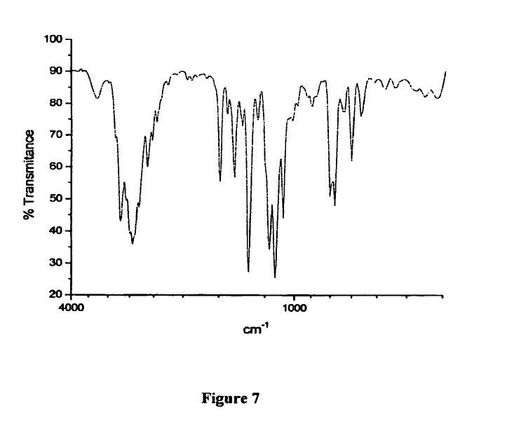

Amorphous cinacalcet hydrochloride is characterized by its XRD pattern as shown in FIG. 6. FIG. 7 illustrates the infrared spectrum of amorphous cinacalcet hydrochloride. Amorphous cinacalcet hydrochloride is further characterized by having a high chemical and optical purity, according to high performance liquid chromatography (HPLC), a low residual solvent content and is generally free of insoluble materials/compounds.

In one embodiment of the invention, amorphous cinacalcet hydrochloride has a degree of chemical purity in the range of about 99.00% to about 99.95% and an optical purity in the range of about 99.0 to about 100%. In another embodiment of the invention, amorphous cinacalcet hydrochloride has a degree of chemical purity in the range of about 99.60% to about 99.80% and an optical purity of about 99.90% to about 100%.

Another aspect of the invention includes a process for preparing cinacalcet hydrochloride Form I, generally comprising:

a. dissolving cinacalcet hydrochloride in an organic solvent;

b. removing the solvent;

c. recovering cinacalcet hydrochloride; and

d. drying the cinacalcet hydrochloride,

wherein the solvent is at least one of an alcoholic solvent, a ketonic solvent, dichloromethane, an ester solvent, an ether solvent, an aprotic solvent or mixtures thereof.

Suitable alcoholic solvents include, but are not limited to, C1 to C4 straight or branched chain alcohol solvents and mixtures thereof (such as methanol, ethanol, n-propanol, 2-propanol, 2-butanol and n-butanol). Preferred alcoholic solvents include, for example, ethanol, 2-propanol and 2-butanol.

Suitable ketonic solvents include, but are not limited to, acetone, metyl ethyl ketone and methyl isopropyl ketone and mixtures thereof. Preferred ketonic solvents include, for example, acetone and methyl ethyl ketone.

Suitable ester solvents include, but are not limited to, ethyl acetate, propyl acetate, butyl acetate, isopropyl acetate. Preferred ester solvents include, for example, ethyl acetate.

Suitable ether solvents include, but are not limited to, diethylether, methyl tert-butyl ether and cyclic ethers such as tetrahydrofuran, 1,4-dioxane, 2-methyltetrahydrofuran, 1,3-dioxolane and mixtures thereof. Preferred ether solvents include, for example, 2-methyltetrahydrofuran and 1,4-dioxane.

Suitable aprotic solvents include, but are not limited to, N,N-dimethylformamide, dimethylsulfoxide, dimethylacetamide, acetonitrile and mixtures thereof. Preferred aprotic solvents include, for example, N,N-dimethylformamide, dimethylsulfoxide and dimethylacetamide.

Preferably, solvent removal is carried out by evaporation at room temperature.

In this process, any of the crystalline forms of cinacalcet hydrochloride may be used.

In another aspect, the invention provides a process for preparing cinacalcet hydrochloride Form I, generally comprising:

a. obtaining cinacalcet hydrochloride by recrystallization from a solvent; and

b. drying the cinacalcet hydrochloride,

wherein the solvent is at least one of an alcoholic solvent, a ketonic solvent, an ester solvent, an ether solvent, a hydrocarbon solvent, an aprotic solvent, water or mixtures thereof.

Suitable alcoholic solvents include, but are not limited to, C1 to C4 straight or branched chain alcohol solvent and mixtures thereof (such as methanol, ethanol, n-propanol, 2-butanol, 2-propanol, 2-butanol and n-butanol). Preferred alcoholic solvents include, for example, 2-propanol, 2-butanol and n-butanol.

Suitable ketonic solvents include, but are not limited to, acetone, methyl ethyl ketone and methyl isopropyl ketone and mixtures thereof. Preferred ketonic solvents include, for example, methyl ethyl ketone and methyl isopropyl ketone.

Suitable ester solvents include, but are not limited to, ethyl acetate, propyl acetate, butyl acetate, isopropyl acetate, isobutyl acetate. Preferred ester solvents include, for example, ethyl acetate, isopropyl acetate, isobutyl acetate and propyl acetate.

Suitable ether solvents include, but are not limited to, diethylether, tert-butyl methyl ether and cyclic ethers such as tetrahydrofuran, 1,4-dioxane, 2-methyl tetrahydrofuran, 1,3-dioxolane and mixtures thereof. Preferred ether solvent include, for example, 1,3-dioxolane.

Suitable hydrocarbon solvents include, but are not limited to, n-pentane, n-hexane and n-heptane and isomers or mixtures thereof, cyclohexane, toluene and xylene and mixtures thereof. Preferred hydrocarbon solvents include, for example, n-heptane and toluene.

Suitable aprotic solvents include, but are not limited to, N,N-dimethylformamide, dimethylsulfoxide, dimethylacetamide, acetonitrile and mixtures thereof. Preferred aprotic solvents include, for example, acetonitrile.

The preferred solvent is a mixture of isobutyl acetate and n-heptane, more preferably isobutyl acetate.

In this process, any of the crystalline forms of cinacalcet hydrochloride may be used.

In another aspect, the invention provides a process for preparing cinacalcet hydrochloride Form I, generally comprising:

a. treating cinacalcet hydrochloride in an organic solvent;

b. recovering the crystalline form as a precipitate; and

c. drying the crystalline form of cinacalcet hydrochloride,

wherein the solvent is at least one of water, ethanol or mixtures thereof.

In this process, any of the crystalline forms of cinacalcet hydrochloride may be used.

In another aspect, the invention provides a process for preparing cinacalcet hydrochloride Form I, generally comprising:

a. dissolving cinacalcet hydrochloride in a first organic solvent,

b. optionally filtering the obtained solution,

c. adding a second solvent, and

d. recovering the crystalline form as a precipitate,

wherein the first organic solvent is at least one of an alcoholic solvent, a ketonic solvent, a chlorinated solvent, an ether solvent or mixtures thereof and the second solvent is at least one of an ether solvent, a hydrocarbon solvent, water or mixtures thereof.

Suitable alcoholic solvents include, but are not limited to, C₁ to C₄ straight or branched chain alcohol solvents and mixtures thereof (such as methanol, ethanol, n-propanol, 2-propanol, 2-butanol and n-butanol). Preferred alcoholic solvent include, for example, methanol, ethanol and 2-propanol.

Suitable ketonic solvents include, but are not limited to, acetone, methyl ethyl ketone and methyl isopropyl ketone and mixtures thereof. Preferred ketonic solvents include, for example, acetone.

Suitable chlorinated solvents include, but are not limited to, dichloromethane, chloroform and mixtures thereof. Preferred chlorinated solvents include, for example, dichloromethane.

Suitable ether solvents include, but are not limited to, diethylether, methyl tert-butyl ether and cyclic ethers such as tetrahydrofuran, 1,4-dioxane, 2-methyl tetrahydrofuran, 1,3-dioxolane and mixtures thereof. Preferred ether solvents include, for example, 1,4-dioxane and tetrahydrofuran as the first organic solvent and methyl ten-butyl ether as the second solvent.

Suitable hydrocarbon solvents include, but are not limited to, n-pentane, n-hexane and n-heptane and isomers or mixtures thereof, cyclohexane, toluene and xylene and mixtures thereof. Preferred hydrocarbon solvents include, for example, n-heptane.

In this process, any of the crystalline forms of cinacalcet hydrochloride may be used.

In another aspect, the invention provides a process for preparing cinacalcet hydrochloride Form II, generally comprising

a. dissolving cinacalcet hydrochloride in chloroform;

b. removing the chloroform;

c. recovering cinacalcet hydrochloride; and

d. drying the cinacalcet hydrochloride,

In this process, any of the crystalline forms of cinacalcet hydrochloride may be used.

In another aspect, the invention provides a process for preparing cinacalcet hydrochloride Form II, generally comprising:

a. suspending cinacalcet hydrochloride in an organic solvent,

b. filtering the obtained solid;

c. recovering cinacalcet hydrochloride; and

d. drying the cinacalcet hydrochloride,

wherein the organic solvent is a chlorinated solvent.

Suitable chlorinated solvents include, but are not limited to, dichloromethane, chloroform and mixtures thereof. Preferred chlorinated solvents include, for example, chloroform.

In this process any of the crystalline forms of cinacalcet hydrochloride may be used.

In another aspect, the invention provides a process for preparing cinacalcet hydrochloride Form III, generally comprising:

a. dissolving cinacalcet hydrochloride in chloroform;

b. adding a second solvent;

c. recovering cinacalcet hydrochloride; and

d. drying the cinacalcet hydrochloride,

wherein the second solvent is at least one of an ether solvent, a hydrocarbon solvent, or mixtures thereof.

Suitable ether solvents include, but are not limited to, diethylether, methyl tert-butyl ether and cyclic ethers such as tetrahydrofuran, 1,4-dioxane, 2-methyl tetrahydrofuran, 1,3-dioxolane and mixtures thereof. Preferred ether solvents include, for example, methyl tert-butyl ether.

Suitable hydrocarbon solvents include, but are not limited to, n-pentane, n-hexane and n-heptane and isomers or mixtures thereof, cyclohexane, toluene and xylene and mixtures thereof. Preferred hydrocarbon solvents include, for example, n-heptane.

In this process, any of the crystalline forms of cinacalcet hydrochloride may be used.

In another aspect, the invention provides processes for preparing amorphous cinacalcet hydrochloride, generally comprising:

a. dissolving cinacalcet hydrochloride in an organic solvent;

b. removing the solvent;

c. recovering cinacalcet hydrochloride; and

d. drying the cinacalcet hydrochloride,

wherein the organic solvent is at least one of an alcoholic solvent, a chlorinated solvent, an ether solvent, a hydrocarbon solvent or mixtures thereof.

Suitable alcoholic solvents include, but are not limited, to C1 to C4 straight or branched chain alcohol solvents and mixtures thereof (e.g., methanol, ethanol, n-propanol, 2-propanol, 2-butanol and n-butanol). Preferred alcoholic solvents include, for example, methanol.

Suitable chlorinated solvents include, but are not limited to, dichloromethane, chloroform and mixtures thereof. Preferred chlorinated solvents include, for example, dichloromethane.

Suitable ether solvents include, but are not limited to, diethylether, methyl tert-butyl ether and cyclic ethers such as tetrahydrofuran, 1 ,4-dioxane, 2-methyl tetrahydrofuran, 1,3-dioxolane and mixtures thereof. Preferred ether solvents include, for example, tetrahydrofuran.

Suitable hydrocarbon solvents include, but are not limited to, n-pentane, n-hexane and n-heptane and isomers or mixtures thereof, cyclohexane, toluene and xylene and mixtures thereof. Preferred hydrocarbon solvents include, for example, toluene.

Preferably, solvent removal is carried out by at least one of evaporation at room temperature and evaporation under vacuum.

In this process, any of the crystalline forms of cinacalcet hydrochloride may be used.

The invention further includes cinacalcet hydrochloride having a particle size distribution wherein approximately 85-95% of the total volume is made of particles having a diameter of approximately 283 μm or below, preferably approximately 85-95% of the total volume is made of particles having a diameter of approximately 80 μm or below, more preferably approximately 85-95% of the total volume is made of particles having a diameter of approximately 35 μm or below.

The invention further includes cinacalcet hydrochloride having a surface area of approximately 0.6 to approximately 2.7 m²/g.

The cinacalcet hydrochloride obtained after recrystallization from heptane-isobutylacetate typically has the following particle size distribution: D₉₀ (v): 200 to 283 μm.

The cinacalcet hydrochloride obtained after recrystallization from isobutylacetate typically has the following particle size distribution: D₉₀ (v): 40 to 80 μm.

The cinacalcet hydrochloride obtained is easily milled. After milling, the cinacalcet hydrochloride obtained typically has the following particle size distribution: D₉₀ (v): 24 to 35 μm.

### SPECIFIC EXAMPLES

The following examples are for illustrative purposes only and are not intended, nor should they be interpreted to, limit the scope of the invention.

General Experimental Conditions:

I. X-ray Powder Diffraction (XRD)

The X-ray diffractograms were obtained using a RX SIEMENS D5000 diffractometer with a vertical goniometer and a copper anodic tube, radiation CuKα, λ=1.54056 Å.

H. Infrared Spectra

Fourier transform infrared spectra were acquired on a Shimadzu FTIR-8300 spectrometer, and polymorphs were characterized in potassium bromide pellets.

III. Thermogravimetric Analysis (TGA)

TGA measurement was carried out in a vented pant at a scan of 10° C./minute from 25.0° C. to 200° C. under a nitrogen purge with a TG-50 available from METTLER-TOLEDO.

IV. Gas Chromatography Method

The gas chromatographic separation was carried out using a RTX-50, 30 m×0.32 mm×0.25 μm column, a head pressure of 10 psi and helium as the carrier gas. Temperature program: 100° C. (0 minute)-20° C./minute-300° C. Injector temperature: 200° C.; Detector (FID) temperature: 300° C.

V. HPLC Methods

a. HPLC Method A

Column: Purospher RP18e (55 mm×4.6 mm×3 um). Eluents: Acetonitrile: Phosphate buffer (pH=2.5). Gradient: 20:80 (2 minutes)-5 minutes-80:20 (3 minutes)-1 minute-20:80 (4 minutes). Detection: UV 220 nm.

b. HPLC Method B

Column: Chiralpak AD. Eluents: 2-propanol (0.5% TFA): n-hexane (0.5% TFA). Gradient: 2:98 (60 minutes)-10−10:90 (5 minutes)-5-2:98 (20 minutes). Detection: UV 270 nm.

c. HPLC Method C

Column: Symmetry C8 (4.6×250 mm, 5 μm). Eluents: 1.26 g ammonium formate in 1000 mL water, adjusted to pH 7 with ammonium hydroxide: Acetonitrile. Gradient 100:0 (20 minutes)-10 minutes-38:62 (30 minutes)-100:0 (10 minutes)-10 minutes. Detection: UV 225 nm.

d. HPLC Method D

Column: Chiralpak AD-H (4.6×250 mm, 5 μm). Mobile phase: 10:90 2-propanol (0.5% TFA):n-hexane (0.5% TFA). Detection: UV 225 nm.

VI. Particle Size Distribution Method

The particle size for cinacalcet hydrochloride was measured using a Malvern Mastersizer S particle size analyzer with an MS1 Small Volume Sample Dispersion Unit stirred cell. A 300RF mm lens and a beam length of 2.4 mm were used. Samples for analysis were prepared by dispersing a weighed amount of cinacalcet hydrochloride (approximately 60 mg) in 20 mL of sample dispersant, previously prepared by dilution of 1.5 g of Soybean Lecithin to 200 mL with Isopar G. The suspension was delivered drop-wise to a background corrected measuring cell previously filled with dispersant (Isopar G) until the obscuration reached the desired level. Volume distributions were obtained for three times. After completing the measurements, the sample cell was emptied and cleaned, refilled with suspending medium, and the sampling procedure repeated again. For characterization, the values of D₁₀, D₅₀ and D₉₀ (by volume) were specifically listed, each one being the mean of the nine values available for each characterization parameter.

VII. Specific Surface Area Method

The BET (Brunauer, Emmett and Teller) specific surface for Cinacalcet hydrochloride was measured using a Micromeritics ASAP2010 equipment. Samples for analysis were degasified at 140° C. under vacuum for two hours. The determination of the adsorption of N₂ at 77° K was measured for relative pressures in the range of 0.07-0.20 for a weighed amount of sample of about 1g.

### Example 1

### Preparation of Cinacalcet Hydrochloride

Under an argon atmosphere, 1.69 g (9.89 mmol, 1.1 eq.) of (R)-1-naphthylethylamine was added to a solution of 2.0 g (8.93 mmol, GC purity: 90.3%) of 3-(3-trifluoro methylphenyl)propanal in 40 mL of tetrahydrofuran. The resulting clear solution was stirred for 15 minutes, and 2 mL of acetic acid and 3.18 g (15.0 mmol) of sodium triacetoxy borohydride were added. The reaction mixture was stirred for two hours, and the solvent was evaporated under vacuum. The resulting residue was dissolved in 30 mL of dichloromethane, and the resulting solution was washed with 30 mL of 10% sodium carbonate solution. The inorganic layer was extracted with 20 mL of dichloromethane, and the solvent of the collected organic phases was evaporated under vacuum. The obtained crude base (3.17 g, 89%) was then dissolved in 5 mL of ethyl acetate and acidified with hydrochloric acid in diethyl ether. Next, the evaporated crude salt was treated with 2-3 mL of ethyl acetate, and the resulting white crystals were filtered, washed with cold ethyl acetate and dried under vacuum at 40° C. to yield 2.65 g of cinacalcet hydrochloride as a white crystalline powder (Yield: 68.5%).

Analytical data: Melting point (MP): 176.4-177.6° C.; purity (determined in base form by GC): 98.9%; XRD (2θ): Form I, see FIG. 1; IR: see FIG. 2.

### Example 2

### Preparation of Cinacalcet Hydrochloride

To a cooled solution (10° C.) of 19.25 g (112 mmol) of (R)-1-(1-naphthyl) ethylamine, 4.5 mL of acetic acid and 500 mL isobutyl acetate, 150 mL of freshly prepared sodium triacetoxyboro hydride and 25.0 g (124.0 mmol, 96.7%) of 3-(3-trifluoromethyl phenyl)propanal in 100 mL isobutyl acetate were added alternatively within four hours in eight portions, starting with the reducing agent. The borohydride aliquots were added simultaneously, while the aldehyde aliquots were added dropwise over 10 minute periods. Once the additions were complete, the resulting white suspension was stirred for 20 minutes, and then 300 mL of distilled water was added. Next, 100 mL of 10% aqueous sodium carbonate was added dropwise. The organic layer was separated and concentrated to about 250 mL. To the concentrated solution was added 75 mL of 2M aqueous hydrochloric acid followed by 150 mL of heptane while stirring. The precipitated crude product was filtered, washed with heptane, washed with water and dried under vacuum at 40° C. to obtain 38.7 g (79.4%) of cinacalcet hydrochloride as a white crystalline powder.

The product was recrystallized from 200 mL of 2-propanol to obtain 26.07 g (53.5%) of cinacalcet hydrochloride as a white crystalline powder. MP: 177.7-179.5° C.; Chemical purity (HPLC, method A): 99.60%; Optical purity (HPLC, Method B) enantiomeric excess: 100%. The (S)-enantiomer of (R)-cinacalcet hydrochloride was not detected.

The sodium triacetoxyborohydride suspension was prepared as follows: to a suspension of 6.5 g (˜170 mmol) of sodium borohydride in 125 mL of isobutyl acetate, 21.55 mL (22.6 g, 376 mmol) of acetic acid was added dropwise while the temperature was kept between 0-5° C. The obtained white suspension was then stirred below 5° C. for about one hour before being used.

### Example 3

### General Method for Preparing Cinacalcet Hydrochloride Form I by Evaporation

A solution of cinacalcet hydrochloride was obtained in a suitable solvent at the concentration shown in Table 1. The solution was allowed to evaporate slowly at room temperature and the solid obtained was smoothly ground for XRD analysis. The results are summarized in Table 1.

TABLE 1

| | Concentration (in | |
|---|---|---|
| Solvent | volumes) | XRD |
| Acetone | 25 | Form I |
| Ethanol | 5 | Form I |
| 2-Propanol | 35 | Form I |
| Methyl ethyl ketone | 25 | Form I |
| Dichloromethane | 3 | Form I |
| Ethyl acetate | 80 | Form I |
| 2-Butanol | 60 | Form I |
| 2-Methyltetrahydrofuran | 50 | Form I |
| Dimethylformamide | 5 | Form I |
| Dimethylacetamide | 5 | Form I |
| Dimethylsulfoxide | 5 | Form I |
| 1,4-Dioxane | 23 | Form I |

### Example 4

### General Method for Preparing Cinacalcet Hydrochloride Form I by Recrystallization

Cinacalcet hydrochloride was recrystallized at reflux temperature in the solvents and concentrations shown in Table 2. The solution was allowed to cool to room temperature while stirring, and after 1 to 4 hours the solid was filtered and analyzed by XRD. The results are summarized in Table 2.

TABLE 2

| | Concentration (in | |
|---|---|---|
| Solvent | volumes) | XRD |
| Water | 52.5 | Form I |
| Ethyl acetate | 11.5 | Form I |
| 2-Propanol | 4.7 | Form I |
| Methyl ethyl ketone | 6.7 | Form I |
| Acetonitrile | 7.7 | Form I |
| 2-Butanol | 3.3 | Form I |
| Propyl acetate | 8.7 | Form I |
| Methyl isopropyl | 5.7 | Form I |
| ketone | | |
| n-butanol | 1.7 | Form I |
| Toluene | 3.3 | Form I |
| 1,3-dioxolane | 4.7 | Form I |
| Isopropyl acetate | 21.3 | Form I |

### Example 5

### Methods for Preparing Cinacalcet Hydrochloride Form I by Treatments at Room Temperature and at Reflux

### Example 5A

Cinacalcet hydrochloride (0.1 g) was suspended in 10 mL of water at room temperature. The mixture was agitated for 24 hours, and the solid was filtered. The solid was analyzed by XRD and found to be Form I.

Analytical data: XRD (2θ): Form I, substantially identical to FIG. 1

### Example 5B

Cinacalcet hydrochloride (0.15 g) was suspended in 5.8 mL of ethyl alcohol. The mixture was heated at reflux for 1 hour, then was allowed to cool at room temperature while stirring, and the solid was filtered. The solid was analyzed by XRD and found to be Form 1.

Analytical data: XRD (2θ): Form I, substantially identical to FIG. 1.

### Example 6

### Methods for Preparing Cinacalcet Hydrochloride Form I by Precipitation

Cinacalcet hydrochloride was dissolved in a first organic solvent at the temperatures and concentrations indicated in Table 3. When possible, the obtained solution was filtered. Thereafter, a second solvent was added, and the obtained mixture was agitated for 30 minutes. Finally the solid was filtered and analyzed by XRD. The results are summarized in Table 3.

TABLE 3

| | | | Concentration | |
|---|---|---|---|---|
| | | | (in volumes) | |
| | | | (first organic | |
| First Organic | | | solvent:second | |
| Solvent | Second Solvent | Temp. | solvent) | XRD |
| Ethanol | Water | 25° C. | 7:30 | Form I |
| Methanol | Water | 25° C. | 2:20 | Form I |
| Acetone | Water | 25° C. | 30:100 | Form I |
| | (mixture | | | |
| | agitated | | | |
| | for 17 hrs.) | | | |
| 1,4-Dioxane | Water | 25° C. | 13.3 | Form I |
| Acetone | Water | Reflux | 10:26.7 | Form I |
| | | (~56° C.) | | |
| 2-propanol | Water | Reflux | 3.3:13.3 | Form I |
| | | (~82° C.) | | |
| Tetrahydrofuran | Water | 25° C. | 4:20 | Form I |
| Ethanol | Methyl tert- | 25° C. | 6.7:20 | Form I |
| | butyl ether | | | |
| Ethanol | n-Heptane | 25° C. | 6.7:20 | Form I |
| Dichloromethane | Methyl tert- | 25° C. | 3.3:20 | Form I |
| | butyl ether | | | |
| Dichloromethane | n-Heptane | 25° C. | 3.3:20 | Form I |
| Tetrahydrofuran | Methyl tert- | 25° C. | 5:20 | Form I |
| | butyl ether | | | |
| Tetrahydrofuran | n-Heptane | 25° C. | 5:20 | Form I |

### Example 7

### Preparation of Cinacalcet Hydrochloride Form II

Cinacalcet hydrochloride (0.5 g) was dissolved in 5 mL of chloroform at room temperature. The solution was allowed to evaporate at room temperature. The obtained solid was ground, analyzed by XRD and found to be Form II.

Analytical data: XRD (2θ): Form II, see FIG. 3.

### Example 8

### Preparation of Cinacalcet Hydrochloride Form II

Cinacalcet hydrochloride (0.5 g) was suspended in 1.7 mL of chloroform at room temperature for 4 hours. The suspension was then filtered, and the obtained solid was analyzed by XRD and found to be Form II.

Analytical data: XRD (2θ): Form II, substantially identical to FIG. 3.

### Example 9

### Preparation of Cinacalcet Hydrochloride Form II

Cinacalcet hydrochloride (0.2 g) was dissolved in 2 mL of chloroform at room temperature. The solvent was evaporated under vacuum, and the obtained solid was analyzed by XRD and found to be Form II.

Analytical data: XRD (2θ): Form II, substantially identical to FIG. 3.

### Example 10

### Preparation of Cinacalcet Hydrochloride Form III

Cinacalcet hydrochloride (0.1 g) was dissolved in 1 mL of chloroform at room temperature. Then 2 mL of n-heptane was added. The suspension was stirred for 30 minutes and filtered. The obtained solid was analyzed by XRD and found to be Form III.

Analytical data: XRD (2θ): Form III, substantially identical to FIG. 4; TGA: see FIG. 5.

### Example 11

### Preparation of Cinacalcet Hydrochloride Form III

Cinacalcet hydrochloride (0.1 g) was dissolved in 1 mL of chloroform at room temperature. Then 2 mL of methyl tert-butyl ether was added. The obtained suspension was stirred for 30 minutes at room temperature and filtered. The obtained solid was analyzed by XRD and found to be Form III.

Analytical data: XRD (2θ): Form III, substantially identical to FIG. 4.

### Example 12

### Preparation of Cinacalcet Hydrochloride Form III

Cinacalcet hydrochloride (0.2 g) was dissolved in 2 mL of chloroform at room temperature. Then, 4 mL of methyl tert-butyl ether was added. The obtained suspension was stirred for 17 hours at room temperature and filtered. The obtained solid was analyzed by XRD and found to be Form III.

Analytical data: XRD (2θ): Form III, see FIG. 4.

### Example 13

### Preparation of Amorphous Cinacalcet Hydrochloride

Cinacalcet hydrochloride (0.1 g) was dissolved in 0.25 mL of methanol. The solution was allowed to evaporate slowly at room temperature. The obtained solid was analyzed by XRD and found to be amorphous cinacalcet hydrochloride.

Analytical data: XRD (2θ): amorphous, see FIG. 6; IR: see FIG. 7.

### Example 14

### Preparation of Amorphous Cinacalcet Hydrochloride

Cinacalcet hydrochloride (0.2 g) was dissolved in 0.67 mL of dichloromethane. The solvent was evaporated under vacuum, and the obtained solid was dried at 60° C. for 15 minutes. The obtained solid was analyzed by XRD and found to be amorphous cinacalcet hydrochloride.

Analytical data: XRD (2θ): amorphous, substantially identical to FIG. 6.

### Example 15

### Preparation of Amorphous Cinacalcet Hydrochloride

Cinacalcet hydrochloride (0.2 g) was dissolved in 1 mL of tetrahydrofuran. The solvent was evaporated under vacuum, and the obtained solid was dried at 60° C. for 15 minutes. The obtained solid was analyzed by XRD and found to be amorphous cinacalcet hydrochloride.

Analytical data: XRD (2θ): amorphous, substantially identical to FIG. 6.

### Example 16

### Preparation of Amorphous Cinacalcet Hydrochloride

Cinacalcet hydrochloride (0.1 g) was dissolved in 0.5 mL of tetrahydrofuran. The solvent was allowed to evaporate slowly at room temperature. The obtained solid was analyzed by XRD and found to be amorphous cinacalcet hydrochloride.

Analytical data: XRD (2θ): amorphous, substantially identical to FIG. 6.

### Example 17

### Preparation of Amorphous Cinacalcet Hydrochloride

Cinacalcet hydrochloride (0.2 g) was dissolved in 14 mL of toluene. The solvent was evaporated under vacuum and the obtained solid was dried at 60° C. for 15 minutes. The obtained solid was analyzed by XRD and found to be amorphous cinacalcet hydrochloride.

Analytical data: XRD (2θ): amorphous, substantially identical to FIG. 6.

### Example 18

### Preparation of Amorphous Cinacalcet Hydrochloride

Cinacalcet hydrochloride (0.1 g) was suspended in 6 mL of toluene and then filtered. The solvent was allowed to evaporate slowly at room temperature. The obtained solid was analyzed by XRD and found to be amorphous cinacalcet hydrochloride.

Analytical data: XRD (2θ): amorphous, substantially identical to FIG. 6.

### Example 19

### Preparation of Cinacalcet Hydrochloride

In a 1,000 mL, four-necked round-bottomed reaction vessel, purged with nitrogen and equipped with a 500 mL pressure-equalized addition funnel, thermometer and blade impeller, are added (in sequence): sodium triacetoxyborohydride (27.85 g, 131.4 mmol) and 75 mL of isobutyl acetate. The resulting white suspension was stirred and cooled to 0-5° C.

In a separate 500 mL, three-necked round-bottomed reaction vessel, purged with nitrogen and equipped with a 100 mL pressure-equalized addition funnel, thermometer and blade impeller, were added (in sequence) at 0-5° C.: (R)-(+)-1-(1-naphthyl)ethylamine (15.00 g, 87.6 mmol), 75 mL of isobutyl acetate, 3-[3-(trifluoromethyl)phenyl]propanal (17.71 g, 87.6 mmol), and another portion of 75 mL of isobutyl acetate. The resulting pale yellow mixture was stirred for 15 minutes at 0-5° C.

The latter mixture was then added dropwise into the sodium triacetoxyborohydride suspension via a pressure-equalized addition funnel over a period of 30 minutes while maintaining the temperature in the 0-5° C. range. Once the addition was complete, the reaction mixture was stirred for 2 hours at 0-5° C. Deionized water (120 g) was then added dropwise to the stirred mixture while maintaining the temperature below 25° C. The mixture was stirred for a total of 30 minutes at 20-25° C., and subsequently the organic phase was separated. Aqueous sodium chloride solution (120.00 g, 5% w/w) was added to the stirred organic phase at 20-25°C. The mixture was then stirred for a total of 30 minutes, and subsequently the organic phase was separated. The organic phase was concentrated to half its volume by removing 115 mL of isobutyl acetate by distillation under vacuum at a vapor temperature of 30° C. The concentrated organic phase was cooled to 5-10° C. while stirring.

An aqueous hydrochloric acid solution was prepared separately by diluting 11.80 g (10.01 mL, 116.5 mmol) of 36% w/w hydrochloric acid or equivalent with 41.30 g of deionized water. The prepared aqueous hydrochloric acid solution was then added dropwise to the stirred organic phase from the pressure-equalized addition funnel while maintaining the temperature at 5-10° C. This addition resulted in a slight temperature rise and the formation of a white suspension. The white suspension was stirred for 30 minutes at a temperature of 5-10° C. n-Heptane (90 mL) was added to the stirred suspension while maintaining a temperature of 5-10° C. The resultant mixture was then stirred for 1 hour at 5-10° C. The suspension was filtered, and the collected solid was washed with 20 g of deionized water to yield 39.60 g of wet, white crude product. The wet solid was then stirred together with 117 g of deionized water for 1 hour at 20-25° C. The suspension was then cooled to 5-10° C., and stirred at this temperature for an additional 30 minutes. The suspension was filtered, and the collected solid was washed with 20 g of deionized water to yield 36.94 g of wet, white crude product. The wet solid was then dissolved in 100 mL of ethanol at 20-25° C. to give a clear, pale yellow solution. This solution was then filtered to remove any insoluble particles. The resulting filtrate was concentrated by removing 70% of the ethanol by distillation under vacuum at a vapor temperature of 28° C. to give a thick, white pasty solid.

Isobutyl acetate (100 mL) was added to the stirred suspension and was then subsequently removed by distillation. This process was repeated a second time with a second 100 mL aliquot of isobutyl acetate. In this second case, only 70% of the added isobutyl acetate was removed by distillation. Isobutyl acetate (148.32 mL) was added to the stirred suspension and the resulting mixture was heated until dissolution of the suspension occurred. The heat was removed, and the solution was allowed to cool to below 85° C. Thereafter, 61.80 mL of n-heptane were added. The resulting suspension was cooled to 0-5° C. and stirred at this temperature for 1 hour. The suspension was filtered and the collected white solid was washed with 20 mL of isobutyl acetate to yield 28.79 g of wet, white solid. The wet solid was dried at 60° C. under vacuum for 4 hours to yield 22.24 g of dry, white cinacalcet hydrochloride (Overall yield: 64.5%). Chemical purity (HPLC, method C): 99.73%; Optical purity (HPLC, method D) enantiomeric excess: 99.92%.

### Example 20

### Large Scale Preparation of Cinacalcet Hydrochloride

In a 630 L stainless steel reactor (clean, dry and inertised), were added (in sequence): 40.9 Kg of sodium triacetoxyborohydride and 96 Kg of isobutyl acetate. The resulting white suspension was then stirred and cooled to 0-5° C.

In a 630 L glass-lined reactor, clean, dry and inertised, were added (in sequence): 22 Kg of (R)-(+)-1-(1-naphthyl)ethylamine and 96 Kg of isobutyl acetate. The resulting mixture was cooled to 0-5° C. Over the naphthylethylamine solution, 26.0 Kg of 3-[3-(trifluoromethyl)phenyl]propanal and another portion of 96 Kg of isobutyl acetate were added. The resulting pale yellow mixture was then stirred for 15 minutes at a temperature of 0-5° C.

The latter mixture was next transferred to the stainless steel reactor, into the sodium triacetoxyborohydride suspension, over a period of 60 minutes while maintaining the temperature in the 0-5° C. range. Once the addition was complete, the reaction mixture was stirred for 2 hours at a temperature of 0-5° C.

Deionized water (176 Kg) was then added to the stirred mixture, and the temperature was adjusted to 20-25° C. The mixture was then stirred for a total of 30 minutes at 20-25° C., and the organic phase was separated.

A 5% w/w aqueous sodium chloride solution (8.8 Kg Sodium chloride and 167 Kg deionized water), previously prepared in a clean 630 L glass-lined reactor, was added to the stirred organic phase, and the temperature was adjusted to 20-25° C. The mixture was stirred for a total of 30 minutes, and the organic phase was separated.

The organic phase was then transferred into a 630 L glass-lined reactor, and the transfer line was washed with 5 Kg of isobutyl acetate. The organic phase was then concentrated to half its volume by removing 159±10 Kg of isobutyl acetate by distillation under vacuum without exceeding a product temperature of 45° C. A white suspension was observed during the final stages of the distillation. The concentrated organic phase was then cooled to 5-10° C. while stirring.

Separately, an aqueous hydrochloric acid solution was prepared in a 100 L glass-lined reactor by diluting 6.2 Kg of 100% eq. w/w hydrochloric acid with 61 Kg of deionized water. The solution was cooled down to 5-10° C. The prepared aqueous hydrochloric acid solution was then transferred to the stirred organic phase while maintaining the temperature at 5-10° C. The white suspension was then stirred for 30 minutes at a temperature of 5-10° C. n-Heptane (90 Kg) was added to the stirred suspension while maintaining a temperature of 5-10° C. The resultant mixture was stirred for 1 hour at a temperature of 5-10° C.

The suspension was next filtered through an 800 mm stainless steel centrifuge equipped with a polypropylene bag. The solid was washed with 25 Kg of deionized water to yield 45.94 Kg of wet, white crude product.

The wet solid was then loaded into a 630 L glass lined reactor together with 172 Kg of deionized water, and stirred for 1 hour at 20-25° C. The suspension was then cooled to 5-10° C., and stirred at this temperature for an additional 30 minutes. The suspension was then filtered through an 800 mm stainless steel centrifuge equipped with a polypropylene bag. The solid was washed with 25 Kg of deionized water to yield 42.27 Kg of wet, white crude product.

The wet solid was loaded into a 630 L glass-lined reactor and dissolved in 115 Kg of ethanol at 20-25° C. to give a clear, pale yellow solution. This solution was then filtered through a plate filter to remove any insoluble particles and transferred to a 630 L clean stainless steel reactor. The transfer line was then washed with 8 Kg of ethanol.

The resulting filtrate was concentrated by removing 90 Kg of the ethanol by distillation under vacuum without exceeding 40° C. product temperature. Filtered isobutyl acetate (126 Kg) was then added to the stirred suspension, and then was subsequently removed by distillation under vacuum without exceeding 40° C. product temperature. This process was repeated a second time with another 126 Kg of filtered isobutyl acetate. In this second case, only 94 f 5 kg of the added isobutyl acetate was removed by distillation.

Next, 189 Kg of filtered isobutyl acetate was added to the stirred suspension, and the resulting mixture was heated to reflux. The suspension was stirred until complete dissolution occurred. The solution was cooled to 75-85° C., and 62 Kg of filtered n-heptane was added. The resulting suspension was cooled to 0-5° C., and stirred at this temperature for 1 hour. The suspension was then filtered through an 800 mm stainless steel centrifuge equipped with a polypropylene bag. The solid was washed with 20 Kg of filtered isobutyl acetate to yield 38.47 Kg of wet, white crude product. The cinacalcet hydrochloride obtained had the following particle size distribution: D₉₀ (v): 263 μm.

The solid was then re-crystallised in a 630 L stainless steel reactor with 215 Kg filtered isobutyl acetate. The resulting mixture was then heated to reflux, and the suspension was stirred until complete dissolution occurred. The solution was cooled to 0-5° C. and stirred at this temperature for 1 hour. Next, the suspension was filtered through an 800 mm stainless steel centrifuge equipped with a polypropylene bag. The solid was washed with 20 Kg of filtered isobutyl acetate to yield 35.98 Kg of wet, white crude product. The wet solid was then dried in a 100 L vacuum paddle drier at 60±5° C. under vacuum for 6 hours to yield 31.23 Kg of dry, white cinacalcet hydrochloride. The cinacalcet hydrochloride obtained had the following particle size distribution: D₉₀ (v): 47 μm.

The dried solid was then milled through a stainless steel pin mill at 14,000 rpm and sieved through a 500 μm sieve to give 29.29 Kg of milled solid. The solid was blended for 2 hours in a 100 L drum blender to give 29.20 Kg of dry, white cinacalcet hydrochloride (Overall yield: 57.6%). The cinacalcet hydrochloride obtained had the following particle size distribution: D₉₀ (v): 24 μm.

It will be apparent to those skilled in the art that various modifications and variations can be made in the present invention and specific examples provided herein without departing from the spirit or scope of the invention. Thus, it is intended that the present invention covers the modifications and variations of this invention that come within the scope of any claims and their equivalents.

What is claimed is:

1. A process for preparing cinacalcet, its salts and solvates thereof, comprising performing a reductive amination, in the absence of titanium isopropoxide, of 3-(3-trifluoromethy phenyl)propanal (Compound III) with (R)-(1-naphthyl)ethylamine (Compound II) to yield cinacalcet.

2. The process of claim 1, wherein said reductive amination comprises the use of sodium triacetoxyborohydride.

3. The process of claim 1, further comprising converting the cinacalcet into one of its corresponding salts and/or solvates thereof.

4. The process of claim 3, wherein said salt of cinacalcet is cinacalcet hydrochloride.

5. The process of claim 1, wherein said cinacalcet hydrochloride is at least one of Form I cinacalcet hydrochloride, Form II cinacalcet hydrochloride, Form III cinacalcet hydrochloride and amorphous cinacalcet hydrochloride.

6. The process of claim 1, wherein Compound III is used in its bisulfite adduct form.

7. The process of claim 1, wherein Compound II is of high optical purity.

8. The process of claim 7, wherein Compound II has an enantiomeric excess of at least 99.5%.

9. Cinacalcet and corresponding pharmaceutically acceptable salts and/or solvates thereof prepared by the process of claim 1.

10. The cinacalcet and corresponding pharmaceutically acceptable salts and/or solvates thereof of claim 9, wherein said cinacalcet and corresponding pharmaceutically acceptable salts thereof has a purity of approximately 99% to approximately 99.95% as measured by high performance liquid chromatography.

11. The cinacalcet and corresponding pharmaceutically acceptable salts and/or solvates thereof of claim 10, wherein said cinacalcet and corresponding pharmaceutically acceptable salts thereof has a purity of approximately 99.6% to approximately 99.8% as measured by high performance liquid chromatography.

12. The cinacalcet and corresponding pharmaceutically acceptable salts and/or solvates thereof of claim 9, wherein said cinacalcet and corresponding pharmaceutically acceptable salts thereof has an optical purity of approximately 99% to approximately 100% as measured by high performance liquid chromatography.

13. The cinacalcet and corresponding pharmaceutically acceptable salts and/or solvates thereof of claim 12, wherein said cinacalcet and corresponding pharmaceutically acceptable salts thereof has a purity of approximately 99.9% to approximately 100% as measured by high performance liquid chromatography.

14. The cinacalcet, its salts and/or solvates of claim 9, wherein said cinacalcet, its salts and/or solvates is at least one of Form I cinacalcet hydrochloride, Form II cinacalcet hydrochloride, Form III cinacalcet hydrochloride and amorphous cinacalcet hydrochloride.

15. The cinacalcet, its salts and/or solvates thereof of claim 9, wherein said cinacalcet, its salts and/or solvates thereof have an enantiomeric excess of at least 99.5%.

16-56. (canceled)
